(19) United States
(12) Patent Application Publication
(10) Pub. No.: US 2003/0175333 A1
(43) Pub. Date: Sep. 18, 2003

(54) Invisible patch for the controlled delivery of cosmetic, dermatological, and pharmaceutical active ingredients onto the skin

(76) Inventors: Adi Shefer, East Brunswick, NJ (US); Samuel Shefer, East Brunswick, NJ (US)

Correspondence Address: Diane Dunn McKay, Mathews, Collins, Shepherd & McKay, P.A., Suite 306, 100 Thanet Circle, Princeton, NJ, 08540, US

(21) Appl. No.: 10/376,736

(22) Filed: Feb. 28, 2003

Related U.S. Application Data
(63) Continuation-in-part of application No. 10/091,935, filed on Mar. 6, 2002.

(51) Int. Cl. A61K 31/715; A61K 9/70

(52) U.S. Cl. 424/449; 514/61

(57) ABSTRACT

The present invention relates to a patch for controlled topical or transdermal delivery of effective levels of cosmetic, dermatological, and pharmaceutical active ingredients onto the skin, hair follicles, and sebaceous glands, with minimal discomfort and ease of use. The patch can be transparent or clear and comprises a rate-controlling matrix layer. The matrix layer comprises water-sensitive, bioadhesive, film forming polymers, a water soluble oligomer, and a surfactant. The cosmetic, dermatological, and pharmaceutical active ingredients are soluble or dispersed in the matrix. The patch becomes tacky when wetted and adheres onto the skin. The adhesive properties of the patch are sufficient to maintain the patch in place on the skin for the recommended treatment period while allowing the patch to be readily removed without causing skin irritation or leaving adhesive residue on the skin.

This application is a continuation in part of U.S. application Ser. No. 10/091,935, filed Mar. 6, 2002, entitled “A Patch for Controlled Delivery of Cosmetic, Dermatological, and Pharmaceutical Active Ingredients into the Skin,” the contents of which are each incorporated by reference into this application.

### BACKGROUND OF THE INVENTION

1. Field of the Invention

The present invention relates to an invisible patch for the controlled delivery of cosmetic, dermatological, and pharmaceutical active ingredients onto the skin which is formed of a single matrix layer. The patch is applied onto the skin by wetting or moistening the target area. Upon application onto the skin surface, the patch dissolves or disintegrates and provides a substantive therapeutic layer to the treatment site over an extended period of time.

2. Description of the Related Art

The localized treatment of body tissues, diseases, and wounds requires that the particular active ingredient be maintained at the site of treatment for an effective period of time. Transdermal patches for the administration of active ingredients onto the skin have become very popular in recent years. These patches adhere to the targeted area and the active ingredient is continually absorbed through the skin into the bloodstream for systemic distribution.

The term “transdermal” as used herein, means transdermal or percutaneous administration, i.e. application of the skin treating composition directly to the skin to be treated. Hence the terms “skin,” “derma,” “epidermis,” and the like shall also be used interchangeably unless specifically stated otherwise.

Transdermal patches, which permit the controlled release of the active ingredients onto the skin, are known from the literature. Two types of patches for skin applications are described in the literature. The first type of patches has a multilayer structure, where the active ingredients are dissolved or dispersed in the various layers. The second type of patch is a pressure-sensitive adhesive patch, where the active is dissolved or dispersed in the patch adhesive layer. Multilayer patches normally have a structure comprising several successive layers in the following order: a first support layer, which is typically occlusive, such as, composed of a material impermeable to the active compound, so as to prevent the evaporation thereof and facilitate transdermal migration; a second storage layer fastened to the support layer and containing the active compound and capable of placement directly in contact with the skin; a layer of an adhesive material applied to the surface of the storage layer and permeable to the active compound to facilitate attachment of the patch to the skin; and a detachable protective layer which hermetically covers the storage layer so as to protect it from any external contamination during storage prior to use of the patch. In the pressure sensitive adhesive patches, the bioactive substances are mixed with and formulated into a pressure sensitive adhesive matrix which may be subsequently coated as a single pressure sensitive adhesive layer.

U.S. Pat. No. 6,280,764 discloses a patch for topical application of an anti-acne formulation has in various embodiments a backing film, a release layer and at least one adhesive polymeric matrix layer located between the backing film and the release layer. The anti-acne formulation is uniformly distributed throughout one or more polymeric matrix layers and has an anti-acne effective amount of at least two agents selected from the group of an anti-microbial, an antiseptic, an anti-irritant, a keratolytic agent, a hormone, a hormone agonist and a hormone antagonist.

U.S. Pat. No. 6,296,869 discloses a dermal patch which includes a substrate formed of a hydrophobic and hydrophilic fiber mixture, and a hydrogel adhesive deposited onto the substrate. The adhesive contains an alpha or beta hydroxy acid. The patch is applied to skin for treating the signs of aging, especially around areas of the eye.

U.S. Pat. No. 6,280,765 discloses patch comprising a hydrophobic polymer layer bound to a support layer and containing: a) first particles of at least one water-soluble active compound, b) second particles of oil, c) at least one liposoluble active compound, d) third particles of a water-absorbing agent all of which are dispersed homogeneously in the polymer layer. This patch allows the packaging and controlled administration of an assembly of skin-nourishing and/or skin-repairing substances of different nature, and also has excellent adhesive power on the skin.

U.S. Pat. No. 5,232,702 describes a patch structure consisting of an occlusive support layer and a polymer layer bound to the support layer. The polymer layer is formed of a matrix of a silicone polymer including, in the dispersed state, fatty substances and hydrophilic active compounds. This form of patch is more particularly suitable for delivering water-soluble active compounds of lipophilic nature.

U.S. Pat. No. 5,976,565 discloses a patch for topical application of an anti-acne formulation has in various embodiments a backing film, a release layer and at least one adhesive polymeric matrix layer located between the backing film and the release layer. The anti-acne formulation is uniformly distributed throughout one or more polymeric matrix layers and has an anti-acne effective amount of at least two agents selected from the group consisting of an anti-microbial, an antiseptic, an anti-irritant, a keratolytic agent, a hormone, a hormone agonist and a hormone antagonist.

U.S. Pat. No. 5,100,672 discloses a pressure sensitive adhesive transdermal patch having a composite adhesive layer reinforced with a web layer. Cosmetically bioactive substances used in the patch include water soluble vitamins such as vitamin C, and liposoluble vitamins A and E or their derivatives.

U.S. Pat. No. 6,180,133 discloses an anti-wrinkle skin treating composition comprises a pressure sensitive matrix patch having dissolved in the adhesive a mixture of antioxidants in the form of a vitamins C ester and vitamin E. Also preferably dissolved in the adhesive are glycerine and a polydiorganosiloxane adhesion-adjusting agent. Optionally dissolved in the adhesive is also one or more members selected from the group consisting of moisturizing agents, skin collagen synthesis promoting agents and exfoliating agents. When applied to a wrinkled skin area the composition acts to diminish fine wrinkles and improves the overall thickness, elasticity, firmness and smoothness of the skin. The modified adhesive properties of the patch are sufficient to maintain the patch in place on the skin for the recommended treatment period while allowing the patch to be readily removed without causing skin irritation or leaving adhesive residue on the skin.

EP-A-0 346 211 describes the use of a copolymer of 2-ethyl-hexyl acrylate and N-vinyl-2-pyrrolidone without absorption promoters. EP-A-0 272 918 describes the use of a macroporous foam in which active ingredient is present in immobilized form. EP-A-0 409 383 describes an estrogen-containing patch in the concentration range from 0.01 to 1% of an estrogen in combination with a water-insoluble vinyl-pyrrolidone for retarded release of the active ingredient to the skin.

U.S. Pat. No. 4,994,267 describes a mixture of a synthetic or natural rubber in combination with an ethylene/vinyl acetate copolymer and acrylate. AU-A-91.76 582 (JP SN 90.202 409) describes the use of an acrylate adhesive in combination with a polyester carrier film. EP-A-0 416 842 describes the use of acrylate copolymers without absorption promoters, which contain active ingredients, preferably oestrogens or norethisterone or norethisterone acetate, by themselves or in combination. These above-described patches are merely carriers of drugs, which allow no control over absorption. Multilayer structured patches are relatively thick, and are therefore fairly uncomfortable on the skin. Furthermore, their appearance and their thickness do not enable the user to wear them in discreet manner.

It is desirable to provide a more aesthetically pleasing, more comfortable, and less obtrusive topical patch for delivering cosmetic, dermatological, and pharmaceutical active ingredients onto the skin which may be applied to sensitive skin sites, such as around the eye.

### SUMMARY OF THE INVENTION

The present invention provides a single layer patch formed of a water soluble matrix comprising a bioadhesive water sensitive polymer, a water soluble oligomer, and a surface active material for delivering cosmetic, dermatological, and pharmaceutical active ingredients onto the skin, hair follicles, and sebaceous glands. The patch dissolves or disintegrates upon contact with skin moisture. The patch of the present invention provides ease of handling and application to the treatment site, comfort, and minimal foreign body sensation. Other preferred characteristics of the patch of the present invention include instantaneous adhesion to the surface upon application; increased residence time for the protection of the affected tissue or the delivery of the active ingredients; and ease of removal of the patch from the affected tissue or natural dissolution of the patch at the delivery site. The patch can further comprise a detachable protective layer to protect the patch from any external contamination during storage prior to use of the patch. Methods for treating the skin surfaces, hair follicles, and sebaceous glands, by applying the patch to the treatment site for the delivery cosmetic, dermatological, and pharmaceutical active ingredient, are also provided. An article of manufacture, such as an invisible bandage, can comprise the patent of the present invention.

### DETAILED DESCRIPTION OF THE INVENTION

The present invention relates to a novel patch to deliver cosmetic, dermatological, and pharmaceutical active ingredients onto the skin, hair follicles, and sebaceous glands. The patch can be translucent or invisible. Upon application and adherence of the patch to the surface of skin, the cosmetic, dermatological, and pharmaceutical active ingredients, diffuse, or penetrate the surrounding tissues, and provide effective delivery to the treatment site. The patch of the present invention offers the advantages of an effective residence time with minimal discomfort and ease of use, and is an appropriate vehicle for local as well as systemic delivery of active ingredients.

Upon application, the patch adheres to the skin surface and holds in place. Water absorption softens the patch, diminishing and eliminating any foreign body sensation. As the patch rests on the skin, delivery of the active ingredients is provided. Residence times can vary, depending on the formulation and materials used. The residence times can be modulated between about a minute to about 24 hours. In addition to providing controlled delivery, once the patch adheres to the surface, it also provides protection to the treatment site, acting as an adhesive bandage. The dissolution rate of the patch in water can be adjusted by selection of polymers used in the patch.

In accordance with the teachings of the present invention the patch comprises a single layer water soluble matrix comprising one or more water sensitive bioadhesive polymers, a water soluble oligomer, and a surfactant. The characteristics of the matrix compositions of the present invention, i.e., dissolution rate, and release rate are dependent in part on the characteristic of individual materials of the composition, in terms of water solubility, crystallinity, and ratio between the polymers, the oligomers, and the surfactants. The use of water-soluble materials and the ability to control water solubility of the patch eases the application of the patch onto the skin and allows for the removal of the patch from the skin by rinsing the site with water.

Bioadhesive Water Sensitive Polymers

Suitable water sensitive bioadhesive polymers include carbohydrates, such as starch derived from different plant sources, including high amylose and high amylopectin varieties. The term “starch,” as referred to herein, is also meant to include water soluble film forming polymeric materials derived from starch including starch derivatives such as starch hydrolyzate products, modified starches, modified starch derivatives and maltodextrins. Other bioadhesive, water soluble polymers for use in the present invention are cellulose and its derivatives, polysaccharide gums and their derivatives, polyethylene glycol, water soluble acrylics, water soluble polyesters, hydroxyalkyl starches, polyvinyl pyrrolidone cellulose derivatives, casein, gelatin, solubilized proteins, polyacrylamide, polyamines, polyquaternary amines, styrene maleic anhydride (SMA) resins, polyethylene amine and any other conventional water soluble polymer or a combination thereof of the above-described materials.

Examples of synthetic water sensitive bioadhesive polymers which are useful for the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble celluloses include water sensitive hydroxyalkyl and carboxyalkyl celluloses such as hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister Pennsylvania. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the Du Pont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides of the non-sweet, coloidally-soluble types, such as natural gums, for example, gum arabic, starch derivatives, dextrinized and hydrolyzed starches, and the like. A suitable polysaccharide is a water dispersible, modified starch commercially available as Capule®, N-Lok®), Hi-Cap™ 100 or Hi-Cap™ 200 commercially available from the National Starch and Chemical Company of Bridgewater, N.J. and Pure-Cote™, commercially available from the Grain Processing Corporation of Muscatine, Iowa. Gum arabic is commercially available from TIC Gums Inc. Belcamp, Midland.

Combinations of different polymers or similar polymers with definite molecular weight characteristics can be used in order to achieve preferred film forming capabilities, mechanical properties, and kinetics of dissolution.

Water Soluble Oligomers

Suitable water soluble oligomers include xylose, ribose, glucose, mannose, galactose, fructose, dextrose, polydextrose, sucrose, maltose, corn syrup solids, palatin, sorbitol, xylitol, mannitol, maltitol, lactitol, xanthan, maltodextrin, galactomanan, tragacanth, manitol, lactitol, oligisaccharides and hydrocolloids and mixtures thereof. Suitable maltodextrins are Maltrin™ M100, Maltrin™ M150, and Maltrin™ M180, commercially available from the Grain Processing Corporation of Muscatine, Iowa, and Lactitol commercially available from the Purac Corporation and Cultor Food Science of Ardsley, N.Y.

Surface Active Agent

Surfactants which can be used in the present invention as a solubility augmenting agent generally include all pharmaceutically-acceptable surfactants, in which the surfactant has an HLB value of at least 10, and preferably at least about 15. Discussions of HLB numbers and how they are determined for specific surfactants can be found in, for example, the publication of ICI Surfactants entitled The HLB System and, in particular, in Chapter 7 of that publication entitled “How to Determine HLB of an Emulsifier” (ICI Americas, Inc., Wilmington, Del., 1992).

In certain embodiments, the HLB value of the surfactant is from about 15 to 50, and in other embodiments the HLB value is from about 15.6 to about 40. Suitable pharmaceutically-acceptable anionic surfactants include, for example, those containing carboxylate, sulfonate, and sulfate ions. Those containing carboxylate ions are sometimes referred to as soaps and are generally prepared by saponification of natural fatty acid glycerides in alkaline solutions. Cations associated with these surfactants include sodium, potassium, ammonium and triethanolamine. The chain length of the fatty acids range from 12 to 18. Although a large number of alkyl sulfates are available as surfactants, a preferred surfactant is sodium lauryl sulfate, which has an HLB value of about 40.

Sodium lauryl sulfate is a water-soluble salt, produced as a white or cream powder, crystals, or flakes. Also known as dodecyl sodium sulfate, sodium lauryl sulfate can be a mixture of sodium alkyl sulfates consisting chiefly of sodium lauryl sulfate. Sodium lauryl sulfate is also known as sulfuric acid monododecyl ester sodium salt. Furthermore, sodium lauryl sulfate is readily available from commercial sources such as Sigma or Aldrich in both solid form and as a solution. The solubility of sodium lauryl sulfate is about 1 gm per 10 ml/water. The fatty acids of coconut oil, consisting chiefly of lauric acid, are catalytically hydrogenated to form the corresponding alcohols. The alcohols are then esterified with sulfuric acid (sulfated) and the resulting mixture of alkyl bisulfates (alkyl sulfuric acids) is converted into sodium salts by reacting with alkali under controlled conditions of pH.

Surfactants can be used in the patch of the present invention such as those selected from the anionic, cationic, nonionic, amphoteric, zwitterionic and combinations thereof. Nonionic and amphoteric surfactants are preferred due to their mildness. Examples of suitable amphoterics are cocoamidopropylbetaine and lauroamphoacetate. Examples of suitable nonionics are dialkylamine oxides, alkyl polyglycosides and methyl glucamides. Examples of mild anionic surfactants include salts of sarcosinate, taurate and cocoyl isethionate. Other surfactants that can be used in the patch of the present invention are sucrose distearate, diglyceryldistearate, tetraglyceryl tristearate, decaglyceryl decastearate, diglyceryl monostearate, hexaglyceyl tristearate, decaglyceryl pentastearate, sorbitan monostearate, sorbitan tristearate, diethylene glycol monostearate, the ester of glycerol and of palmitic acid and stearic acid, monostearate polyoxyethylenated containing 2 oxyethylene units, glyceryl mono- and dibehenate and pentaerythrityl tetrastearate.

Alternative anionic surfactants for use as surface active agents in the present invention include docusate salts such as the sodium salt thereof. Other suitable anionic surfactants include, without limitation, alkyl carboxylates, acyl lactylates, alkyl ether carboxylates, N-acyl sarcosinates, polyvalent alkyl carbonates, N-acyl glutamates, fatty acid, polypeptide condensates and sulfuric acid esters.

In other aspects of the invention amphoteric (amphipathic/amphiphilic surfactants), non-ionic surfactants and/or cationic surfactants can be used as the surface active agent in the coprocessed compositions of the present invention. Suitable pharmaceutically-acceptable non-ionic surfactants include, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters and simethicone. The HLB for one acceptable non-ionic surfactant, polysorbate 40, is about 15.6.

Other suitable pharmaceutically-acceptable surfactants include acacia, benzalkonium chloride, cholesterol, emulsifying wax, glycerol monostearate, lanolin alcohols, lecithin, poloxamer, polyoxyethylene, and castor oil derivatives.

Solubilizers can also be used in the present invention including glycerol, propylene glycol, polyalcohols, sorbitol and sorbitol derivatives.

The amount of surfactants and solubilizers used in the patch of the present invention can each independently range from about 0.01 to about 45%, preferably from about 0.1 to about 30%, most preferably from about 1 to about 20% by weight.

Active Ingredients

The active substances to be released by the patch can serve the dermal treatment of local skin diseases, the intradermal and transdermal treatment of diseases, the treatment of wounds, or the skin care in cosmetic preparations.

The patch can include one or more cosmetic, dermatological, and pharmaceutical active ingredients that have an effect on the skin, including, but not limited to: anti-oxidants; free radical scavengers; moisturizers; depigmentation agents; reflectants; humectants; antimicrobial (e.g., antibacterial) agents; allergy inhibitors; anti-acne agents; anti-aging agents; anti-wrinkling agents, antiseptics; analgesics; antitussives; antipruritics; local anesthetics; anti-hair loss agents; hair growth promoting agents; hair growth inhibitor agents, antihistamines; keratolytic agents; anti-inflammatory agents; fresheners; healing agents; anti infectives; inflammation inhibitors; anticholinergics; vasoconstrictors; vasodilators; wound healing promoters; peptides, polypeptides and proteins; deodorants and antiperspirants; skin emollients and skin moisturizers; hair conditioners; hair softeners; hair moisturizers; tanning agents; skin lightening agents; antifungals such as antifungals for foot preparations; depilating agents; external analgesics; counterirritants; hemorrhoidals; insecticides; poison ivy products; poison oak products; burn products; anti-diaper rash agents; prickly heat agents; make-up preparations; vitamins; amino acids and their derivatives; herbal extracts; retinoids; flavoids; sensory markers (i.e., cooling agents, heating agents, etc.); skin conditioners; hair lighteners; chelating agents; cell turnover enhancers; coloring agents; sunscreens; anesthetics; immunomodulators and nourishing agents; moisture absorbers; sebum absorbers and the like, and mixtures thereof.

Local anaesthetics, local antibiotics, antiseptics, antimycotics, antihistaminics, and antipruritic drugs; keratolytics and caustic drugs; virustatics, antiscabietic agents, steroids, as well as different substances for the treatment of acne, psoriasis, photodermatoses, or precancerous stages can be used with the patch of the present invention for the dermal treatment of local skin diseases. Active substances applicable by the intradermal route with the patch of the present invention include, for example, steroid and non-steroid antirheumatics, local anaesthetics, substances stimulating the blood flow, vasoprotectors and vasoconstrictors to treat vascular diseases, as well as active substances to influence processes in the subcutaneous fatty tissue. Transdermally applicable active substances to be used in the patch of the present invention include, for example, analgesics, anti-arrhrythmic drugs, narcotics and their antagonists, neuroleptics, hormones or hormone substitutes, antidepressants, tranquilizers, hypnotics, psychostimulants, antiparkinson drugs, ganglionic blockers, sympathomimetics, alpha-sympatholytics, beta-sympatholytics, antisympathotonics, anti-asthmatics, antiemetics, appetite depressants, diuretics, or active substances for weight reduction, and the like. Because of the small thickness of the system according to the present invention preferred active substances are those developing their action already at very low concentrations. Examples of these preferred active substances include steroids, such as estradiol, estriol, progesterone, norethisterone, norethindrone, levonorgestrel and their derivatives, as well as estradiol diacetate, norgestamate, gestagens, desogestrel, demegestrone, promegestrone, testosterone, hydrocortisones and their derivatives; nitro compounds, such as amyl nitrate, nitroglycerin, isosorbide dinitrate; amine compounds, such as nicotine, chlorpheniramine, terfenadine, and triprolidine; oxicam derivatives such as piroxicam; mucopolysaccharases such as thiomucase; opioid substances such as buprenorphine, morphine, fentanyl and their salts, derivatives or analogues, naloxone, codeine, dihydroergotamine, lysergic acid derivatives, pizotiline, salbutamol, terbutaline; prostaglandins, such as PGA, PGB, PGE and the PGF-series, for example, misoprostol and enprostil, omeprazol, imipramine; benzamides, such as metoclopramines and scopolamine; peptides and growth factors such as EGF, TGF, PDGF, and the like; somatostatin; clonidin; dihydropyridines, such as nifedipine, nitrendipine, verapamil, diltiazem, ephedrine, propanolol, metoprolol, spironolactone; thiazides such as hydrochlorothiazide and flunarizine. Styptic active substances and wound-cleansing substances, such as enzymes, antiseptics, disinfectants, and antibiotics; pain-relieving agents and anaesthetic active substances, as well as active substances promoting wound healing to stimulate granulation, to induce vascularization, or to promote epithelization can be used with the patch of the present invention for the treatment of wounds.

The patch of the present invention can also comprise a steroid hormone, preferably estradiol either alone or combined with other drugs, which is used in transdermal application for hormone substitution during postmenopause or for the treatment of osteoporosis. The patch of the present invention including estradiol can also be applied on long-term wounds, for instance crural ulcera, for the treatment of wounds.

The patch of the present invention can also comprise vegetable preparations, such as extracts or tinctures for the treatment of topical skin diseases. Suitable extracts or tinctures include oak bark extract, walnut extract, tincture of arnica, hamamelis extract, ribwort extract, pansy extract, thyme or sage extract; for the treatment of damaged or injured skin, for example, St. John's wort tincture, cone flowers tincture, chamomile flowers extract, or calendula flowers tincture; and for the care of exhausted and damaged skin, for example, birch leaves extract, nettle extract, coldsfoot extract, comfrey tincture, horsetail extract, or aloe vera extract. Vegetable preparations can also be released from the film layer for the intradermal treatment of diseases, for example, extracts of horse chestnut and butcher's broom in case of vein diseases, or extracts and tinctures of arnica, calendula, and capsicum in case of contusions, distortions, or haemorrhages. Vegetable preparations in the system according to the present invention may also be used in transdermal therapy, for example, ginseng extract in case of geriatric complaints; valerian tincture, extracts of melissa and hop to cause a sedative effect in case of superexcitation, sleep disturbances, and stress; extracts of kola and tea to achieve a stimulative effect; or hawthorn extract to stabilize the circulatory system.

Suitable effervescent agents that can be used with the patch of the present invention include sodium bicarbonate and sodium carbonate.

Suitable amino acid agents that can be used with the patch of the present invention include amino acids derived from the hydrolysis of various proteins as well as the salts, esters, and acyl derivatives thereof. Examples of such amino acid agents include amphoteric amino acids such as alkylamido alkylamines, stearyl acetyl glutamate, capryloyl silk amino acid, caprylol collagen amino acids; capryloyl kertain amino acids; capryloyl pea amino acids; cocodimonium hydroxypropyl silk amino acids; corn gluten amino acids; cysteine; glutamic acid; glycine; hair keratin amino acids; hair amino acids such as aspartic acid, threonine, serine, glutamic acid, proline, glycine, alanine, half-cystine, valine, methionine, isoleucine, leucine, tyrosine, phenylalanine, cysteic acid, lysine, histidine, arginine, cysteine, tryptophan, citrulline; lysine; silk amino acids, wheat amino acids; and mixtures thereof

Suitable peptides, polypeptides, and proteins that can be used with the patch of the present invention include those polymers that have a long chain, such as at least about 10 carbon atoms, and a high molecular weight, such as at least about 1000, and are formed by self-condensation of amino acids. Examples of such proteins include collagen, deoxyribonuclease, iodized corn protein; keratin; milk protein; protease; serum protein; silk; sweet almond protein; wheat germ protein; wheat protein; wheat protein, alpha and beta helix of keratin proteins; hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Examples of suitable vitamins that can be used with the patch of the present invention include vitamin B complex; including thiamine, nicotinic acid, biotin, pantothenic acid, choline, riboflavin, vitamin B6, vitamin B 12, pyridoxine, inositol, carnitine; vitamins A, C, D, E, K and their derivatives such as vitamin A palmitate and pro-vitamins, such as panthenol (pro vitamin B5) and panthenol triacetate, and mixtures thereof.

Examples of suitable antibacterial agents that can be used with the patch of the present invention include bacitracin, erythromycin, neomycin, tetracycline, chlortetracycline, benzethonium chloride, phenol, and mixtures thereof.

Examples of suitable skin emollients and skin moisturizers that can be used with the patch of the present invention include mineral oil, lanolin, vegetable oils, isostearyl isostearate, glyceryl laurate, methyl gluceth 10, methyl gluceth 20 chitosan, and mixtures thereof.

Examples of suitable hair conditioners that can be used with the patch of the present invention include quaternized compounds such as behenamidopropyl PG-dimonium chloride, tricetylammonium chloride, dihydrogenated tallowamidoethyl hydroxyethylmonium methosulfate, and mixtures thereof as well as lipophilic compounds like cetyl alcohol, stearyl alcohol, hydrogenated polydecene, and mixtures thereof.

Examples of sunscreen agents that can be used with the patch of the present invention include butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, padimate o, red petrolatum, and mixtures thereof. An example of a suitable tanning agent that can be used with the patch of the present invention is dihydroxyacetone. Examples of suitable skin lightening agents that can be used with the patch of the present invention include hydroquinone, catechol and its derivatives, ascorbic acid and its derivatives, and mixtures thereof.

Examples of suitable insecticides that can be used with the patch of the present invention (including insect repellents, anti-scabies and anti-lice treatments) include permethrin, pyrethrin, piperonyl butoxide, imidacloprid, N,N-diethyl toluamide, which refers to the material containing predominantly the meta isomer.

An example of a suitable anti fungal for foot preparations that can be used with the patch of the present invention includes tolnaftate.

Examples of suitable depilating agents that can be used with the patch of the present invention include calcium thioglycolate, magnesium thioglycolate, potassium thioglycolate, strontium thioglycolate, and mixtures thereof.

Examples of suitable external analgesics and local anesthetics that can be used with the patch of the present invention include benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof

Examples of suitable antiperspirants and deodorants that can be used with the patch of the present invention include aluminium chlorohydrates, aluminium zirconium chlorohydrates, and mixtures thereof.

Examples of suitable counterirritants that can be used with the patch of the present invention include camphor, menthol, methyl salicylate, peppermint and clove oils, ichtammol, and mixtures thereof

An example of a suitable inflammation inhibitor that can be used with the patch of the present invention includes hydrocortisone.

Examples of suitable hemorrhoidal products that can be used with the patch of the present invention include anesthetics such as benzocaine, pramoxine hydrochloride, and mixtures thereof; antiseptics such as benzethonium chloride; astringents such as zinc oxide, bismuth subgallate, balsam Peru, and mixtures thereof; skin protectants such as cod liver oil, vegetable oil, and mixtures thereof.

A type of benefit agent that can be used with the patch of the present invention includes those therapeutic agents that are effective in the treatment of dandruff, seborrheic dermatitis, and psoriasis as well as the symptoms associated therewith. Examples of such suitable therapeutic agents include zinc pyrithione, shale oil and derivatives thereof such as sulfonated shale oil, selenium sulfide, sulfur; salicylic acid; coal tar; povidone-iodine and imidazoles.

Antimicrobials that can be used with the patch of the present invention for topical application are penicillins, cephalosporins, other beta-lactam compounds, aminoglycosides, tetracyclines, erythromycin, antifungal agents, and the like and a combination thereof.

Antiseptics that can be used with the patch of the present invention for topical application onto acneiform skin are triclosan (Irgasan DP 300), phenoxy isopropanol, resorcinol, chlorhexidine, povidone and iodine.

Keratolytic agents that can be used with the patch of the present invention for topical application onto acneiform skin are salicylic acid, benzoyl peroxide, sulphur, retinoic acid and any of a number of fruit acids and alpha hydoxy acids.

Anti-irritants that can be used with the patch of the present invention for the topical application onto acneiform skin are alpha-bisabolol, farnesol, chamomile extract and glycyrrhetinic acid.

Examples of anti-inflammatory analgesic agents that can be used with the patch of the present invention include acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexarnac, piroxicam, phenylbutazone, oxyphenbutazone, clofezone, pentazocine, mepirizole, tiaramide hydrochloride, and the like. Examples of steroidal anti-inflammatory agents that can be used with the patch of the present invention include hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, and the like.

Examples of antihistamines that can be used with the patch of the present invention include diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, and the like. Examples of local anesthetics that can be used with the patch of the present invention include dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, and the like.

Examples of bactericides and disinfectants that can be used with the patch of the present invention include thimerosal, phenol, thymol, benzalkonium chloride, benzethonium chloride, chlorhexidine, povidone iode, cetylpyridinium chloride, eugenol, trimethylammonium bromide, and the like. Examples of vasoconstrictors that can be used with the patch of the present invention include naphazoline nitrate, tetrahydrozoline hydrochloride, oxymetazoline hydrochloride, phenylephrine hydrochloride, tramazoline hydrochloride, and the like. Examples of hemostatics that can be used with the patch of the present invention include thrombin, phytonadione, protamine sulfate, aminocaproic acid, tranexamic acid, carbazochrome, carbaxochrome sodium sulfanate, rutin, hesperidin, and the like.

Examples of chemotherapeutic drugs that can be used with the patch of the present invention include sulfamine, sulfathiazole, sulfadiazine, homosulfamine, sulfisoxazole, sulfisomidine, sulfamethizole, nitrofurazone, and the like. Examples of antibiotics that can be used with the patch of the present invention include penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, and the like.

Examples of antiviral drugs that can be used with the patch of the present invention include protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Example of cosmetic active ingredients that can be used with the patch of the present invention are D-alpha-tocopherol, DL-alpha-tocopherol, D-alpha-tocopheryl acetate, DL-alpha-tocopheryl acetate, ascorbyl palmitate, vitamin F and vitamin F glycerides, vitamin D, vitamin D₂, vitamin D₃, retinol, retinol esters, retinyl palmitate, retinyl propionate, beta-carotene, D-panthenol, famesol, farnesyl acetate; jojoba oils and blackcurrant oils rich in essential fatty acids; 5-n-octanoylsalicylic acid and esters thereof, salicylic acid and esters thereof; alkyl esters of .alpha.-hydroxy acids such as citric acid, lactic acid, glycolic acid; asiatic acid, madecassic acid, asiaticoside, total extract of Centella asiatica, beta-glycyrrhetinic acid, alpha-bisabolol, ceramides such as 2-oleoylamino-1,3-octadecane; phytanetriol, phospholipids of marine origin which are rich in polyunsaturated essential fatty acids, ethoxyquine; extract of rosemary, extract of balm, quercetin, extract of dried microalgae, anti-inflammatory agents, such as steroidal anti-inflammatory agents, and biostimulants, for example hormones or compounds for the synthesis of lipids and/or proteins.

Alpha-Hydroxy acids (AHAs) can be used in the patch of the present invention as exfoliants, moisturizers, and emollients. Lactic acid salts can be used in the patch of the present invention such as sodium lactate, and can be hypothesized to be part of the skin's own natural moisturizing system. In addition, AHAs and salicylic acid can be used in the patch of the present invention as a structurally similar beta-hydroxy acid as peeling agents. The moisturizing activity of AHAs and their ability to exfoliate the skin and interfere with intercellular cohesion in the outer epidermis is well known. It has been suggested that AHAs interfere with cohesion in the stratum granulosum, unlike salicylic acid and other exfoliants.

Vitamin C (ascorbic acid) can be used in the patch of the present invention. Vitamin C promotes collagen (connective tissue) synthesis, lipid (fat) and carbohydrate metabolism, and the synthesis of neurotransmitters. It is also essential for optimum maintenance of the immune system. Vitamin C is toxic to a wide range of cancer cells, especially melanoma. The oxidizing enzyme tyrosine that catalyzes the aerobic action of tyrosine into melanin and other pigments is also inhibited by the presence of vitamin C. Vitamin C has been found to be effective in catalyzing the immune response to many viral and bacterial infections. Besides the many applicable uses set forth above, vitamin C is essential for collagen synthesis and wound healing. The patch of the present invention can comprise a combination of vitamin C, vitamin E and other ingredients, such as moisturizers, collagen synthesis promoting agents and exfoliating agents.

Skin treating compositions can be used in the patch of the present invention. Skin treating compositions can comprise vitamin C, vitamin E, and optionally, alpha-hydroxy acids, such as lactic and glycolic acids and other keratinolytics for the treatment or prevention of wrinkles and skin dryness.

According to the present invention the patch can also be marked in the form of colors, letters, numbers, dates, codes, pictographs and the like by means of screen printing. The film layer of the patch can be dyed by means of soluble dyes or pigments. Alternatively, the patch can be completely transparent or invisible on the skin.

The patch can be used as any product applied to the skin where it is desired that the product blend in with the wearer's skin or be completely transparent so as to be invisible. The patch can be used as an invisible bandage to promote healing and tissue regeneration after application to the skin.

Skin conditioners, moisturizers and surfactants can be included as additives in the patch of the present invention. Illustrative conditioners include mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

The concentration of the active ingredient in the patch of the present invention depends on the desired treatment strength. Typically, this concentration can range from about 0.001% to about 80% by weight relative to the total weight of the oily phase. Preferably, this percentage is in the range of about 1% to about 50%.

Plasticizers, penetration enhancer, as described in the text “Transdermal Delivery of Drugs, A. F. Kydonieus (ED) 1987 CRL Press and in U.S. Pat. Nos. 4,913,905, 4,917,676 and 5,032,403 hereby incorporated by reference into this application, coloring agents, and preservatives can be included in the patch of the present invention and comprise no more than about 10% of the final weight of the patch, but the amount can vary depending on the active ingredient or other components. Glycerin, which is also a moisturizing agent, can be added as an anti-irritant or to modulate the delivery of the other skin treating agents and can be present in amounts of from about 0 to about 20% by weight.

The patch of the invention can also contain encapsulated active ingredients in water sensitive or hydrophobic controlled release systems in the form of nano-spheres and micro-spheres. The encapsulated active ingredients are dispersed homogeneously in the polymeric film. Examples of encapsulated active ingredients in water sensitive micro-spheres are spray dried active ingredients with starch and other natural or synthetic water-soluble polymers. On contact with skin moisture, the spray dried micro-spheres, comprising the active ingredients, are released, thereby promoting the controlled delivery or the enhanced bioavailability of active ingredients and minimizing the interaction of active ingredients with the other compounds present in the patch. Examples of encapsulated ingredients in nano-spheres are dispersions of hydrophobic materials, such as lipids, waxes, and hydrophobic polymers comprising active ingredients in the hydrophobic matrix. On contact with skin moisture, the hydrophobic nano-spheres, comprising the active ingredients, are released, thereby promoting the controlled delivery or the enhanced bioavailability of active ingredients and minimizing the interaction of active ingredients with the other compounds present in the patch.

Water Sensitive Micro-Spheres

Water sensitive micro-spheres can be incorporated in the compositions and articles of the present invention by mixing the microspheres with a water sensitive material before dispersing the microspheres in the matrix composition.

Water-sensitive materials to encapsulate active ingredients in the present invention comprise water soluble and water dispersible natural oligomers, synthetic oligomers, natural polymers, synthetic polymers and copolymers, starch derivatives, oligosaccharide, polysaccharides, hydrocolloids, natural gums, proteins, and mixtures thereof.

Suitable water sensitive materials to encapsulate ingredients of the present invention include xylose, ribose, glucose, mannose, galactose, fructose, dextrose, polydextrose, sucrose, maltose, or corn syrup solids, palatin, sorbitol, xylitol, mannitol, maltitol, lactitol, xanthan, maltodextrin, galactomanan or tragacanth, and mixtures thereof. Water sensitive materials also include oligosaccharides and hydrocolloids.

Examples of synthetic water sensitive polymers which are useful to encapsulate ingredients of the present invention in the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful to encapsulate ingredients of the present invention in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the Du Pont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides to encapsulate ingredients of the present invention of the non-sweet, coloidally-soluble types, such as natural gums, for example, gum arabic, starch derivates, dextrinized and hydrolyzed starches, and the like. A suitable polysaccharide is a water dispersible, modified starch commercially available as Capule®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment the natural gum is a gum arabic, commercially available from TIC Gums Inc. Belcamp, Midland. Suitable hydrocolloids are xanthan, maltodextrin, galactomanan or tragacanth, preferably maltodextrins such as Maltrin™ M100, and Maltrin™ M150, commercially available from the Grain Processing Corporation of Muscatine, Iowa.

In one embodiment, the water sensitive micro-spheres can be bioadhesive. Bioadhesive micro-sphere can be created by incorporating a bioadhesive material into the micro-sphere matrix.

The water-sensitive micro-spheres of the present invention comprising active ingredients can be prepared by the steps of (1) forming an aqueous phase of the moisture sensitive materials (either a single material or mixture of several materials); (2) emulsifying the active ingredients in the aqueous phase; and (3) removing moisture to create free-flowing powder. For example, moisture can be removed by spray drying droplets of emulsion. Spray drying is well known in the art and been used commercially in many applications, including foods where the core material is a flavoring oil and cosmetics where the core material is a fragrance oil, as described in Cf. Balassa, “Microencapsulation in the Food Industry”, CRC Critical Review Journal in Food Technology, July 1971, pp 245-265; Barreto, “Spray Dried Perfumes for Specialties, Soap and Chemical Specialties”, December 1966; Maleeny, Spray Dried Perfumes, Soap and San Chem, January 1958, pp. 135 et seq.; Flinn and Nack, “Advances in Microencapsulation Techniques”, Batelle Technical Review, Vo. 16, No. 2, pp. 2-8 (1967); U.S. Pat. Nos. 5,525,367; and 5,417,153 which are incorporated herein as references.

The micro-spheres have size of from about 0.5 micron to about 300 microns, more preferably from about 1 micron to about 200 microns, most preferably from about 2 microns to about 30 microns. The present invention preferably has minimal active agents on the surface of the spheres, preferably less than about 1%.

Hydrophobic Nano-Spheres Encapsulated in Water Sensitive Micro-Spheres

Multi component carrier systems, comprising of solid hydrophobic nano-spheres encapsulated in a moisture, water, or pH sensitive micro-sphere, can also be incorporated in the compositions and devices of the present invention by mixing them with the water sensitive materials before dispersing them in the composition. These multi component systems provides moisture-triggered release of the actives that are encapsulated in the micro-sphere matrix, as well as, prolong release of the actives encapsulated that are encapsulated in the nano-sphere matrix over an extended period of time. The surface properties of the nano-spheres may be modified to enhance the affinity of the nano-spheres for a particular residue expressed on a cell surface or their affinity for a cell surface protein or receptor. Active ingredients can be incorporated in the hydrophobic nano-spheres, in the water, or pH sensitive micro-spheres, or in both the nano and micro-spheres. The deposition of the nano-spheres onto the target surface is improved by optimizing particle size to ensure entrainment of the particles within target surface and by modifying their surface to enhance the affinity of the nano-spheres for a particular residue expressed on a cell surface or their affinity for a cell surface protein or receptor to maximize interaction between the particles and the target surface.

With respect to the interaction between the particles and the target surface, various chemical groups and bioadhesive materials can be incorporated in the nano-spheres structure, depending on the target surface. A cationic surface active agent will create positively charged nano-spheres; an anionic surface active agent will create negatively charged nano-spheres; a nonionic surface active will create neutral charged nano-spheres; and a zwitterionic surface active agent will create a variable charged nano-spheres.

In one embodiment, the nano-spheres of the present invention are bioadhesive. Bioadhesive nano-sphere can be created by incorporating a bioadhesive material into the solid hydrophobic matrix of the nano-spheres, by incorporating bioadhesive material in the pH sensitive micro-sphere matrix, or by using a bioadhesive material in the nano-sphere matrix in conjunction with bioadhesive material in the micro-sphere matrix.

These multi component systems are in the form of free-flowing, powder, having the advantages of:

(i) protection of the active ingredients, during storage, or until needed and reaches the target site;

(ii) water, or pH triggered release of the first said active ingredient and the nano-spheres comprising the second said active ingredient in response to moisture or in response to change in pH in the system proximate environment, and,

(iii) site specific targeted delivery and enhanced deposition of active ingredients, onto the target surface;

(iv) enhanced bioavelability of active ingredients encapsulated in the nano-spheres; and

(v) prolonged release of active ingredients, over an extended period of time.

A method for producing the multi component controlled release system including active ingredients comprises the steps of:

(i) incorporating the active ingredients into the solid hydrophobic nano-spheres;

(ii) forming an aqueous mixture comprising of one or more active agents, the nano-spheres, and a water, or pH or sensitive materials; and

(iii) spray drying the mixture to form a dry powder composition.

A process for producing the multi component controlled release system including the active ingredients comprises the steps of:

(i) heating hydrophobic materials to a temperature above the melting point of the materials to form a melt;

(ii) dissolving or dispersing the first active agent into the melt, and optionally a targeting material;

(iii) dissolving or dispersing a second active agent, the water or pH sensitive material, and optionally a targeting material, in the aqueous phase;

(iv) heating the composition to above the melting temperature of the hydrophobic materials;

(v) mixing the hot melt with the aqueous phase to form a dispersion;

(vi) high shear homogenization of the dispersion at a temperature above the melting temperature until a homogeneous fine dispersion is obtained having a sphere size of from about 1 micron to about 2 microns;

(vii) cooling the dispersion to ambient temperature; and

(viii) spray drying the emulsified mixed suspension to form a dry powder composition.

The hydrophobic matrix sustains the diffusion rate of the pharmacotherapeutic active ingredients, through the nano-spheres and enables them to be released onto the target site over an extended period of time. The micro-spheres have an average sphere size in the range from about 20 microns to about 100 microns. The nano-sphere have an average sphere size in the range from about 0.01 micron to about 5 microns and having a melting point in the range from about 30 degrees C. to about 90 degrees C.

Nano-spheres formed of a hydrophobic material provide a controlled release system in order to release the active agent over an extended period of time by molecular diffusion. Active agents in the hydrophobic matrix of the nano-spheres can be released by transient diffusion. The theoretical early and late time approximation of the release rate of the active ingredients dissolved in the hydrophobic matrix of the nano-spheres can be calculated from the following equations:

Early time approximation

(mₜ/m_{sec})<0.4

$$\begin{matrix}\frac{{M}_{t}}{{M}_{∞}} = 4 {( \frac{{D}_{p} t}{Π {r}^{2}} )}^{1 / 2} - \frac{{D}_{p} t}{{r}^{2}} & ( 1 ) \\ \frac{ⅆ {M}_{t} / {M}_{∞}}{ⅆ t} = 2 {( \frac{{D}_{p}}{Π {r}^{2} t} )}^{1 / 2} - \frac{{D}_{p}}{{r}^{2}} & ( 2 )\end{matrix}$$

Late time approximation

(mₜ/m₂₈)>0.6

$$\begin{matrix}\frac{{M}_{t}}{{M}_{∞}} = 1 - \frac{4}{{( 2.405 )}^{2}} exp ( \frac{- {( 2.405 )}^{2} {D}_{p} t}{{r}^{2}} ) & ( 3 ) \\ \frac{ⅆ {M}_{t} / {M}_{∞}}{ⅆ t} = 1 - \frac{4 {D}_{p}}{{r}^{2}} exp ( \frac{- {( 2.405 )}^{2} {D}_{p} t}{{r}^{2}} ) & ( 4 )\end{matrix}$$

wherein:

r is the radius of the cylinder,

m ∞ is the amount fragrance released from the controlled release system after infinite time;

mₜ is the amount fragrance released from the controlled release system after time t; and

Dₚ is the diffusion coefficient of the fragrance or aroma chemical in the matrix. The release rate for releasing the active agents from the hydrophobic nano-spheres is typically slower than the release rate for releasing active agent from the water or pH sensitive matrix. The active agents can be selected to be incorporated into either the hydrophobic nano-spheres or the water or pH sensitive matrix depending on the desired time for release of the active agents. For example, the water or pH sensitive matrix formed in accordance with the present invention can release the first active agent at a predetermined pH to provide a “burst” with continued release of the first active agent and nano-spheres formed in accordance with the present invention can release the active agent depending on the release rate from an initial time such as within few days, up to a period of few weeks.

The patch of the present invention can be prepared by numerous methods known in the art. In one embodiment, the components are dissolved in an appropriate solvent or combination of solvents to prepare a solution. Solvents for use in the present invention comprise water, methanol, ethanol, or low alkyl alcohols such as isopropyl alcohol, acetone, or dichloroethane, alone or combination. The solvent can also be used as a plasticizer or dissolution-rate-modifying agent. The patch may consist of a detachable protective layer to protect the patch from any external contamination during storage prior to use of the patch.

The patch of the present invention can be applied to human skin using hands by wetting the patch or the targeted site. The patch becomes tacky when wetted, and adheres onto the skin. The adhesive properties of the patch are sufficient to maintain the patch in place on the skin for the recommended treatment period while allowing the patch to be readily removed without causing skin irritation or leaving adhesive residue on the skin. The patch can be removed by rinsing the area with water, thus requiring less force than other conventional pressure-sensitive adhesive patches.

The patch of the present invention can include a detachable protective layer to protect the patch from external contamination during storage prior to use of the patch. The protective layer can be formed of plastic or paper.

The primary active ingredients to be delivered to the skin are preferably cosmetic, dermatological, and pharmaceutical and can be a single agent or can comprise a mixture of active ingredients.

In order to ensure that the patch is simple and comfortable to use, a suitable size and thickness of a single patch has been identified. The patch of the present invention can be produced in a variety of sizes dependent on the area to be treated. The size of the patch is classified as a small patch being about 0.5 to about 2 cm² and a large patch up to about 40 cm². Typically, the size of the patch is from about 0.5 to about 3 cm² and preferably about 2 cm². The patch can be made in a variety of shapes and can be substantially transparent or clear, a flesh-like color or shade so as to effectively blend with the skin of wearer and appears invisible or translucent. The patches according to the present invention can be cut according to an appropriate contour corresponding to the region of skin surface to be treated, for example in the form of a mask for application to the face, especially for application around the eyes, on the bags under the eyes or on the forehead. The patch according to the present invention can be cut into any other shape required for application to a defined region of the body. In general, the size of a patch in accordance with the invention is between about 0.25 cm² to about 500 cm². A patch intended for the depigmentation of pigmented skin blemishes can be small in size, less than about 1 cm². For example, a patch with a slimming action can have a large surface area, which is sufficient to cover part of a thigh. The patches cut to a desired size and shape can be used on a surface of skin to be treated by applying them directly to the skin after the targeted area has been wetted.

The thickness of the patch can have a range from about 10 microns to about 1000 microns, and more preferably from about 50 to about 250 microns.

The invention also provides a method for the use of the patch to deliver agents to the skin. The method generally comprises wetting the patch, or the target surface and applying the patch to the skin. The patch can be removed from the skin by washing the area with water.

The invention can be further illustrated by the following examples preferred embodiments thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

Preparation of a Patch for Acne Treatment

Compositions used in the preparation of a patch for the topical treatment of acne and acnei form skin diseases are described in Table 1-4. The examples were conducted using salicylic acid, as keratolytic agent, in an amount of 0.1 to 2% w/w together with an anti-irritant such as alpha-bisabolol in 0.01 to 3% w/w, an antiseptic such as triclosan (Irgasan DP 300) in 0.1 to 1% w/w, ascorbic acid (Vitamin C), vitamin E, and a solubilizer such as sorbitan monooleate in 0.1 to 5% w/w. Both ascorbic acid and vitamin E are useful in the topical treatment of acne.

TABLE 1

| | QUANTITY |
|---|---|
| COMPONENT | % w/w (on a dry basis) |
| Hydroxypropyl Cellulose | 86.5 |
| alpha-Bisabolol¹ | 1.0 |
| Irgasan DP 300² | 0.3 |
| Salicylic acid | 0.2 |
| Polysorbate 20 | 5 |
| Maltrin 100 | 5 |
| sorbitan monooleate | 2 |

¹alpha-Bisabolol is 6-methyl-2-(4-methyl-3-cyclohexen-1-yl)-5-hepten-2-ol
²Irgasan DP 300 is 2,4,4′-trichloro-2′-hydroxy diphenyl ether

TABLE 2

| | QUANTITY |
|---|---|
| COMPONENT | % w/w (on a dry basis) |
| Hydroxypropyl Cellulose | 74.8 |
| Salicylic acid | 0.2 |
| Ascorbic Acid | 10 |
| Polysorbate 20 | 5 |
| Maltrin 100 | 5 |
| Spray dried particles of Vitamin E | 5 |

TABLE 3

| | QUANTITY |
|---|---|
| COMPONENT | % w/w (on a dry basis) |
| Polyvinyl Alcohol | 65.8 |
| Polyvinyl Pyrrolidone | 15 |
| Glycerin | 4 |
| Salicylic acid | 0.2 |
| Ascorbic Acid | 10 |
| Vitamin E | 5 |
| Polysorbate 20 | 5 |

TABLE 4

| | QUANTITY |
|---|---|
| COMPONENT | % w/w (on a dry basis) |
| Gantrez ®¹ S-97 BF | 75.8 |
| Glycerin | 4 |
| Salicylic acid | 0.2 |
| Ascorbic Acid | 10 |
| Vitamin E | 5 |
| Green Tea Extract | 5 |

¹Gantrez ® S-97 BF is 2-Buttenedioic Acid (Z)-, Polymer with Methoxyethene (commercially available from the ISP Technologies, Inc. of Wayne, New Jersey)

The patch was cut into a circular shape with nominal size of 1 cm² and thickness of 150 microns. The target area on the skin was wetted and the patch was applied.

### Example 2

Preparation of a Patch for Skin Lightening

Compositions used in the preparation of a patch for skin lightening that contains an inhibitor of tyrosinase activity, phytolight®, as skin lightening agent (a mixture of botanical extracts, Coletica Inc., Northport N.Y.) are described in Table 5-6.

TABLE 5

| | QUANTITY |
|---|---|
| COMPONENT | % w/w (on a dry basis) |
| Hydroxypropyl Cellulose | 70 |
| Polysorbate 20 | 5 |
| Lactitol | 10 |
| phytolight ® | 5 |
| Ascorbic Acid | 5 |
| Vitamin E | 5 |

TABLE 6

| | QUANTITY |
|---|---|
| COMPONENT | % w/w (on a dry basis) |
| Polyvinyl Alcohol | 80 |
| Polyvinyl Pyrrolidone | 10 |
| Polysorbate 20 | 5 |
| phytolight ® | 5 |
| Ascorbic Acid | 5 |

The patch was cut into a circular shape with nominal size of 1 cm² and thickness of 150 microns. The target area on the skin was wetted and the patch was applied.

### Example 3

Preparation of a Patch to Reduce Eye Puffiness

Compositions used in the preparation of a patch to reduce eye puffiness that contains a stabilized flavonoid extract that stimulate blood circulation and inhibits elastase, flavagrum® PEG, as active agent (a mixture of botanical extracts, Coletica Inc., Northport N.Y.) are described in Table 7-8.

TABLE 7

| | QUANTITY |
|---|---|
| COMPONENT | % w/w (on a dry basis) |
| Hydroxypropyl Cellulose | 75 |
| flavagrum ® PEG | 5 |
| Polysorbate 20 | 5 |
| Maltrin 180 | 10 |
| Lactitol | 5 |

TABLE 8

| | QUANTITY |
|---|---|
| COMPONENT | % w/w (on a dry basis) |
| Polyvinyl Alcohol | 60 |
| Polyvinyl Pyrrolidone | 14 |
| flavagrum ® PEG | 5 |
| cooling agent¹ | 1 |
| Polysorbate 20 | 5 |
| Maltrin 180 | 10 |
| Lactitol | 5 |

¹Cyclohexanecarboxamide, N-Ethyl-5-Methyl-2-(1-Methylethyl)-

The patch was cut into a circular shape with nominal size of 1 cm² and thickness of 150 microns. The target area on the skin was wetted and the patch was applied.

### Example 4

Preparation of a Depilatory Patch

Compositions used in the preparation of hair removal are described in Table 9-11. The examples are conducted using Calcium Thioglycolate or Potassium Thioglycolate as depilatory agents, in an amount of 5 to 20% w/w together with calcium hydroxide or sodium hydroxide in 1 to 10% w/w, Urea as hair swelling agent in 4 to 10% w/w, and Glycerin as plasticizer at 1 to 20% w/w.

TABLE 9

| | QUANTITY |
|---|---|
| COMPONENT | % w/w (on a dry basis) |
| Hydroxypropyl Cellulose | 68 |
| Urea | 4 |
| Sodium Hydroxide | 2 |
| Potassium Thioglycolate | 6 |
| Polysorbate 20 | 5 |
| Maltrin 180 | 10 |
| Lactitol | 5 |

TABLE 10

| | QUANTITY |
|---|---|
| COMPONENT | % w/w (on a dry basis) |
| Polyvinyl Alcohol | 60 |
| Polyvinyl Pyrrolidone | 14 |
| Glycerin | 10 |
| Calcium Thioglycolate | 10 |
| Calcium Hydroxide | 2 |
| Urea | 4 |

TABLE 11

| | QUANTITY |
|---|---|
| COMPONENT | % w/w (on a dry basis) |
| Polyvinyl Alcohol | 25 |
| Polyvinyl Pyrrolidone | 10 |
| Cetyl trimethyl ammonium chloride | 5 |
| Glycerin | 15 |
| Calcium Thioglycolate | 15 |
| Calcium Hydroxide | 5 |
| Urea | 4 |
| Fragrance | 1 |

The patch was cut into a circular shape with nominal size of 1 cm² and thickness of 150 microns. The depilatory patch is applied on the skin surface after wetting the area. The patch is allowed to stand for about 5 to 10 minutes and the strength of hair is reduced or dissolved by the effect of the depilatory agent. Hair can be removed without leaving any residue by washing off the patch from the skin.

### Example 5

Preparation of a Patch for Treating the Signs of Aging

Compositions used in the preparation of a patch for the topical treatment of skin to reduce the signs of aging are described in Table 12-14. The examples were conducted using anti aging and anti oxidants active ingredients such as retinol, ascorbic acid (Vitamin C), Vitamin E, Green Tea Extract.

TABLE 12

| | QUANTITY |
|---|---|
| COMPONENT | % w/w (on a dry basis) |
| Instant Textra ™¹ | 75 |
| Maltrin ™ M100² | 10 |
| Glycerin | 5 |
| Ascorbic acid | 10 |

¹Instant Textra ™ is a food starch-modified (commercially available from the National Starch and Chemical Company of Bridgewater, New Jersey)
²Maltrin ™ M100 (commercially available from the Grain Processing Corporation of Muscatine, Iowa)

TABLE 13

| | QUANTITY |
|---|---|
| COMPONENT | % w/w (on a dry basis) |
| N-Lite ® L¹ | 65 |
| Maltrin ™ M100² | 10 |
| Glycerin | 5 |
| Ascorbic acid | 10 |
| Green Tea Extract | 10 |

¹N-Lite ® L is a food starch-modified (commercially available from the National Starch and Chemical Company of Bridgewater, New Jersey)
²Maltrin ™ M100 (commercially available from the Grain Processing Corporation of Muscatine, Iowa)

TABLE 14

| | QUANTITY |
|---|---|
| COMPONENT | % w/w (on a dry basis) |
| Instant Pure-Cote ® B792¹ | 65 |
| Maltrin ™ M100² | 10 |
| Glycerin | 5 |
| Ascorbic acid | 10 |
| Vitamin E | 5 |

¹Instant Pure-Cote ® B792 is a food starch-modified (commercially available from the Grain Processing Corporation of Muscatine, Iowa)
²Maltrin ™ M100 (commercially available from the Grain Processing Corporation of Muscatine, Iowa)

TABLE 15

| | QUANTITY |
|---|---|
| COMPONENT | % w/w (on a dry basis) |
| Polyvinyl Alcohol | 50 |
| Polyvinyl Pyrrolidone | 15 |
| Polysorbate 20 | 5 |
| Maltrin 180 | 10 |
| Lactitol | 5 |
| Glycerin | 5 |
| Retinol | 10 |

The patch was cut into a circular shape with nominal size of 1 cm² and thickness of 150 microns. The target area on the skin was wetted and the patch was applied.

### Example 6

Preparation of a Patch for Bum Treatment

Compositions used in the preparation of a local anesthetic patch to alleviate pain and discomfort are described in Table 16. The example is conducted using benzocaine.

TABLE 16

| | QUANTITY |
|---|---|
| COMPONENT | % w/w (on a dry basis) |
| Polyvinyl Alcohol | 50 |
| Polyvinyl Pyrrolidone | 15 |
| Polysorbate 20 | 5 |
| Maltrin 180 | 10 |
| Lactitol | 5 |
| Glycerin | 10 |
| Benzocaine¹ | 1.5 |

¹Benzocaine is ethyl 4-aminobenzoate

The benzocaine is a local anesthetic which would alleviate pain and discomfort, and Glycerin is an excellent humectant which moisturizes the skin. The patch was cut into a circular shape with nominal size of 1 cm² and thickness of 150 microns. The target area on the skin was wetted and the patch was applied.

### Example 7

Preparation of a Pain Relief Patch

Composition used in the preparation of a pain relief patch is described in Table 17. The example is conducted using ibuprofen.

TABLE 17

| | QUANTITY |
|---|---|
| COMPONENT | % w/w (on a dry basis) |
| Polyvinyl Alcohol | 50 |
| Polyvinyl Pyrrolidone | 15 |
| Polysorbate 20 | 5 |
| Maltrin 180 | 10 |
| Lactitol | 5 |
| Glycerin | 10 |
| ibuprofen | 5 |

The patch was cut into a circular shape with nominal size of 1 cm² and thickness of 150 microns. The target area on the skin was wetted and the patch was applied.

### Example 8

Preparation of an Antibiotic Patch

Composition used in the preparation of an antibiotic patch to is described in Table 18. The example is conducted using chloramphenicol.

TABLE 18

| | QUANTITY |
|---|---|
| COMPONENT | % w/w (on a dry basis) |
| Polyvinyl Alcohol | 50 |
| Polyvinyl Pyrrolidone | 1 |
| Polysorbate 20 | 5 |
| Maltrin 180 | 10 |
| Lactitol | 5 |
| Glycerin | 10 |
| chloramphenicol | 0.55 |

The patch is useful in the antibiotic treatment of a variety of topical bacterial, chlamydial, and rickettsial infections.

### Example 9

Preparation of Self Tanning Patch

Composition used in the preparation of a self tanning patch to is described in Table 18. The example is conducted using dihydroxyacetone as tanning agent and L-Lysine as tanning accelerator.

TABLE 18

| | QUANTITY |
|---|---|
| COMPONENT | % w/w (on a dry basis) |
| Polyvinyl Alcohol | 50 |
| Polyvinyl Pyrrolidone | 15 |
| Polysorbate 20 | 5 |
| Maltrin 180 | 10 |
| Lactitol | 5 |
| Glycerin | 5 |
| dihydroxyacetone | 5 |
| L-Lysine | 5 |

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. A patch for controlled topical or transdermal delivery of effective levels of cosmetic, dermatological, or pharmaceutical active ingredients onto the skin, hair follicles, or sebaceous glands comprising a single matrix layer formed of a bioadhesive water-sensitive polymer, a water soluble oligomer, a surfactant and one or more cosmetic, dermatological, or pharmaceutical active ingredients.

2. The patch according to claim 1 wherein said patch dissolves or disintegrates upon contact with moisture.

3. The patch according to claim 1 wherein the bioadhesive water-sensitive polymer comprises one or more materials selected from the group consisting of:
a carbohydrate, starch, starch derivatives, starch hydrolyzate, modified starches, modified starch derivatives, hydroxyalkyl starches, hydroxypropyl cellulose, alkyl and carboxyalkyl cellulose, alkali metal salts of carboxyalkyl cellulose, polyvinyl alcohol, cellulose derivatives, polysaccharide, gum arabic and their derivatives, polyethylene glycol, water soluble acrylic, water soluble polyester, polyvinyl pyrrolidone, polyvinyl pyrrolidone cellulose derivatives, casein, gelatin, solubilized proteins, polyacrylamide, polyamine, polyquaternary amine, styrene maleic anhydride resins, polyethylene amine, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxide and water soluble polyamide.

4. The patch according to claim 1 wherein the bioadhesive water-sensitive polymer comprises one or more materials selected from the group consisting of polyvinyl alcohol, polyvinyl pyrrolidone, modified starch derivatives and hydrolyzed starches.

5. The patch according to claim 1 wherein the oligomer comprises one or more materials selected from the group consisting of xylose, ribose, glucose, mannose, galactose, fructose, dextrose, polydextrose, sucrose, maltose, corn syrup solids, palatin, sorbitol, xylitol, mannitol, maltitol, lactitol, xanthan, maltodextrin, galactomanan, tragacanth, manitol, lactitol, oligisaccharides and hydrocolloids.

6. The patch according to claim 1 wherein the surfactant has an HLB of at least about 10.

7. The patch according to claim 1 wherein the surfactant comprises one or more materials selected from the group consisting of anionic, cationic, nonionic, amphoteric, zwitterionic and combinations thereof.

8. The patch according to claim 1 wherein the surfactant comprises one or more materials selected from the group consisting of sodium lauryl sulfate, cocoamidopropylbetaine, lauroamphoacetate, dialkylamine oxide, alkyl polyglycoside, methyl glucamide, sarcosinate, taurate, cocoyl isethionate, sucrose distearate, diglyceryldistearate, tetraglyceryl tristearate, decaglyceryl decastearate, diglyceryl monostearate, hexaglyceyl tristearate, decaglyceryl pentastearate, sorbitan monostearate, sorbitan tristearate, diethylene glycol monostearate, ester of glycerol and of palmitic acid, ester of glycerol and stearic acid, monostearate polyoxyethylenated containing 2 oxyethylene units, glyceryl mono- and dibehenate and pentaerythrityl tetrastearate, alkyl carboxylates, acyl lactylates, alkyl ether carboxylates, N-acyl sarcosinates, polyvalent alkyl carbonates, N-acyl glutamates, fatty acid, polypeptide condensates, sulfuric acid esters, polyoxyethylene, lecithin, ethoxylated alcohols, ethoxylated esters, ethoxylated amides, polyoxypropylene, propoxylated alcohol, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, sorbitan esters, sucrose esters, glucose esters and simethicone.

9. The patch according to claim 1 wherein the one or more of said cosmetic, dermatological, and pharmaceutical active ingredients are uniformly distributed throughout the matrix layer.

10. The patch according to claim 1 wherein the one or more of said cosmetic, dermatological, or pharmaceutical active ingredients are selected from the group consisting of anti-oxidant; free radical scavenger; moisturizer; depigmentation agent; liporegulator; reflectant; humectant; antimicrobial agent; allergy inhibitor; anti-acne agent; anti-aging agent; anti-wrinkling agent; antiseptic agent; analgesic; antitussive; antipruritic; local anesthetic; anti-hair loss agent; hair growth promoting agent; hair growth inhibitor agent; anti-dandruff agent; antihistamine; keratolytic agent; anti-inflammatory agent; freshener; healing agent; anti infective; inflammation inhibitor; anti-emetic; anticholinergic; vasoconstrictor; vasodilator; wound healing promoter; peptide, polypeptide; protein; deodorant; antiperspirant; skin emollient; skin moisturizer; softener; hair conditioner; hair softener; hair moisturizer; tanning agent; skin lightening agent; antifungal; depilating agent; external analgesic; counterirritant; hemorrhoidal; insecticide; poison ivy treatment agent; poison oak treatment agent; burn treatment agent; anti-diaper rash agent; prickly heat agent; make-up preparation; vitamin; amino acid; amino acid derivative; herbal extract; retinoid; flavoid; sensory marker; anti-oxidant; skin conditioner; hair lightener; chelating agent; cell turnover enhancer; coloring agent; sunscreen; anesthetic; immunomodulator, nourishing agent; moisture absorber; sebum absorber and mixtures thereof.

11. The patch according to claim 1 wherein the one or more of said cosmetic, dermatological, or pharmaceutical active ingredients are anti-septic agents selected from the group consisting of triclosan povidone, iodine, resorcinol, phenoxy, isopropanol and chlorhexidine.

12. The patch according to claim 1 wherein the one or more of said cosmetic, dermatological, or pharmaceutical active ingredients are anti-microbial agents selected from the group consisting of erythromyxin, tetracycline, cephalosporin and clindamycin.

13. The patch according to claim 1 wherein the one or more of said cosmetic, dermatological, or pharmaceutical active ingredients are keratolytic agents of salicylic acid.

14. The patch according to claim 1 wherein the one or more of said cosmetic, dermatological, or pharmaceutical active ingredients are topical antiseptics selected from the group consisting of iodine, mercury, silver, phenol, and nitrofurazone and combinations thereof.

15. The patch according to claim 1 wherein the one or more of said cosmetic, dermatological, or pharmaceutical active ingredients are anti-inflammatory agents chosen from the group consisting of aspirin and ibuprofen.

16. The patch according to claim 1 wherein the one or more of said cosmetic, dermatological, or pharmaceutical active ingredients are anti-irritant compositions selected from the group consisting of an antihistamine and calamine.

17. The patch according to claim 1 wherein the one or more of said cosmetic, dermatological, or pharmaceutical active ingredients are counter-irritant compositions selected from the group consisting of capsaicin, menthol, and clove oil.

18. The patch according to claim 1 wherein the one or more of said cosmetic, dermatological, or pharmaceutical active ingredients are moisturizers.

19. The patch according to claim 18 wherein the one or more of said cosmetic, dermatological, or pharmaceutical active ingredients are moisturizers selected from the group consisting of aloe, lanolin, glycerin, mineral oil, and combinations thereof.

20. The patch of claim 1 wherein the one or more of said cosmetic, dermatological, or pharmaceutical active ingredients are permeation enhancers.

21. The patch of claim 1 wherein the one or more of said cosmetic, dermatological, or pharmaceutical active ingredients selected from the group consisting of an anti-inflammatory analgesic agent, a steroid hormone, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, and combinations thereof.

22. The patch of claim 1 wherein the one or more of said cosmetic, dermatological, or pharmaceutical active ingredients are anti-aging active agents.

23. The patch of claim 1 wherein the one or more of said cosmetic, dermatological, or pharmaceutical active ingredients are depigmentation active agents.

24. The patch of claim 1 wherein the one or more of said cosmetic, dermatological, or pharmaceutical active ingredients are anti-acne agents.

25. The patch of claim 1 wherein the one or more of said cosmetic, dermatological, or pharmaceutical active ingredients are tanning agents of dihydroxyacetone.

26. The patch according to claim 1 wherein the one or more of said cosmetic, dermatological, or pharmaceutical active ingredients are effervescent agents selected from the group consisting of sodium bicarbonate and sodium carbonate.

27. A patch according to claim 1 wherein said one or more dermatological active ingredients are selected from the group consisting of anti-oxidants, free radical scavengers, moisturizers, depigmenting agents, liporegulators, anti-acne agents, anti-dandruff agents, anti-aging agents, softeners, anti-wrinkle agents, keratolytic agents, anti-inflammatory agents, fresheners, healing agents, vascular protectors, antibacterial agents, antifungal agents, antiperspirants, deodorants, skin conditioners, anesthetics, immunomodulators and nourishing agents, moisture absorbers, and sebum absorbers.

28. A patch according to claim 1 further comprising a solubilizer selected from the group consisting of glycerol, propylene glycol, polyalcohol, sorbitol and sorbitol derivatives.

29. The patch according to claim 1 wherein said matrix is transparent.

30. The patch according to claim 1 wherein said matrix has a color.

31. The patch according to claim 1 having a size in the range of about 0.25 cm² to about 500 cm², and a shape to match the shape of a region to be treated.

32. The patch according to claim 1 wherein the matrix layer has a thickness from about 0.0001 mm to about 1.0 mm.

33. The patch according to claim 1 wherein said patch further comprises a detachable protective layer.

34. The patch according to claim 1 wherein said one or more cosmetic, dermatological, or pharmaceutical active ingredients are encapsulated in one or more of hydrophobic nanospheres, microspheres or hydrophobic nanospheres encapsulated in microspheres.

35. The patch according to claim 34 wherein said microsphere is formed of a moisture sensitive, water sensitive or pH sensitive material.

36. The patch according to claim 34 wherein said one or more of said cosmetic, dermatological and pharmaceutical active ingredients are encapsulated in said hydrophobic nanospheres of said hydrophobic nanospheres encapsulated in microspheres, in said microsphere of said hydrophobic nanospheres encapsulated in microspheres or in both said hydrophobic nanospheres of said hydrophobic nanospheres encapsulated in microspheres and said microspheres of said hydrophobic nanospheres encapsulated in microspheres.

37. The patch according to claim 34 wherein said microsphere is formed of a water-sensitive material comprising one or more materials selected from water soluble and water dispersible natural oligomers, synthetic oligomers, natural polymers, synthetic polymers and copolymers, starch derivatives, oligosaccharide, polysaccharides, hydrocolloids, natural gums, proteins, xylose, ribose, glucose, mannose, galactose, fructose, dextrose, polydextrose, sucrose, maltose, or corn syrup solids, palatin, sorbitol, xylitol, mannitol, maltitol, lactitol, xanthan, maltodextrin, galactomanan or tragacanth, polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester, water soluble hydroxyalkyl and carboxyalkyl celluloses.

38. The patch according to claim 34 wherein said microsphere has a size from about 0.5 microns to about 300 microns.

39. The patch according to claim 34 wherein said hydrophobic nanosphere is formed of a lipid or wax.

40. The patch according to claim 34 wherein said nanosphere has an average sphere size in the range from about 0.01 micron to about 5 microns.

41. A method for treating the skin comprising the step of:
applying to a surface of the skin to be treated the patch according to claim 1.

42. The method of claim 41 further comprising the step of:
moistening a surface of the skin before the step of applying the patch.

43. The method of claim 41 wherein the one or more of said cosmetic, dermatological, or pharmaceutical active ingredients are selected from the group consisting of anti-oxidants, free radical scavengers, moisturizers, depigmenting agents, liporegulators, anti-acne agents, anti-dandruff agents, anti-aging agents, softeners, anti-wrinkle agents, keratolytic agents, anti-inflammatory agents, fresheners, healing agents, vascular protectors, antibacterial agents, antifungal agents, antiperspirants, deodorants, skin conditioners, anesthetics, immunomodulators and nourishing agents, moisture absorbers, and sebum absorbers.

44. A method for adhering a patch onto the skin, hair follicles or sebaceous glands comprising the steps of:
wetting an area of said skin hair follicles or sebaceous glands; and
affixing the patch to the skin, hair follicles or sebaceous glands, said patch comprising a single matrix layer formed of a bioadhesive water-sensitive polymer, a water soluble oligomer, and a surfactant.

45. The method of claim 44 wherein said patch further comprises one or more cosmetic, dermatological, or pharmaceutical active ingredients which are selected from the group consisting of anti-oxidant; free radical scavenger; moisturizer; depigmentation agent; liporegulator; reflectant; humectant; antimicrobial agent; allergy inhibitor; anti-acne agent; anti-aging agent; anti-wrinkling agent; antiseptic agent; analgesic; antitussive; antipruritic; local anesthetic; anti-hair loss agent; hair growth promoting agent; hair growth inhibitor agent; anti-dandruff agent; antihistamine; keratolytic agent; anti-inflammatory agent; freshener; healing agent; anti infective; inflammation inhibitor; anti-emetic; anticholinergic; vasoconstrictor; vasodilator; wound healing promoter; peptide, polypeptide; protein; deodorant; antiperspirant; skin emollient; skin moisturizer; softener; hair conditioner; hair softener; hair moisturizer; tanning agent; skin lightening agent; antifungal; depilating agent; external analgesic; counterirritant; hemorrhoidal; insecticide; poison ivy treatment agent; poison oak treatment agent; burn treatment agent; anti-diaper rash agent; prickly heat agent; make-up preparation; vitamin; amino acid; amino acid derivative; herbal extract; retinoid; flavoid; sensory marker; anti-oxidant; skin conditioner; hair lightener; chelating agent; cell turnover enhancer; coloring agent; sunscreen; anesthetic; immunomodulator, nourishing agent; moisture absorber; sebum absorber and mixtures thereof.

46. A method of using a patch comprising the step of:
applying a patch to the skin, hair follicles or sebaceous glands for a period of application in a range of about one minute to about 12 hours, said patch comprising a single matrix layer formed of a bioadhesive water-sensitive polymer, a water soluble oligomer, and a surfactant.

47. The method of claim 46 wherein said patch further comprises one or more pharmaceutical active ingredients selected from the group consisting of an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, and a combination thereof.

48. A patch for controlled topical or transdermal delivery of effective levels of cosmetic, dermatological, or pharmaceutical active ingredients onto the skin, hair follicles, or sebaceous glands comprising a matrix layer which comprises one or more materials selected from the group consisting of polyvinyl alcohol, polyvinyl pyrrolidone, modified starch derivatives and hydrolyzed starches and a combination thereof, an oligomer selected from the group consisting of xylose, ribose, glucose, mannose, galactose, fructose, dextrose, polydextrose, sucrose, maltose, corn syrup solids, palatin, sorbitol, xylitol, mannitol, maltitol, lactitol, xanthan, maltodextrin, galactomanan, tragacanth, manitol, lactitol, oligisaccharides and hydrocolloids, a surfactant, and one or more of said cosmetic, dermatological, and pharmaceutical active ingredients uniformly distributed throughout the polymeric water-soluble matrix layer.

49. An article of manufacture applied to the skin comprising the patch of claim 1.

50. The article of claim 49 wherein said article is an invisible bandage.
